(19) United States
(12) Patent Application Publication
(10) Pub. No.: US 2024/0173169 A1
(43) Pub. Date: May 30, 2024

(54) ADJUSTABLE SHUNTING SYSTEMS AND ASSOCIATED SYSTEMS AND METHODS

(71) Applicant: Shifamed Holdings, LLC, Campbell, CA (US)

(72) Inventors: Eric Schultz, Los Altos, CA (US); Katherine Sapozhnikov, Campbell, CA (US); Tom Saul, Portland, OR (US); Michael Drews, Palo Alto, CA (US); Robert Chang, Belmont, CA (US); Leticia Marquez Garcia, Gilroy, CA (US); Richard Lilly, San Jose, CA (US)

(21) Appl. No.: 18/551,571

(22) PCT Filed: Sep. 3, 2021
(86) PCT No.: PCT/US2021/049140

§ 371 Date: Sep. 20, 2023

Related U.S. Application Data
(60) Provisional application No. 63/175,147, filed on Apr. 15, 2021.
(60) Provisional application No. 63/216,801, filed on Jun. 30, 2021.

(51) Int. Cl. A61F 9/007

(52) U.S. Cl. CPC A61F 9/00781; A61F 2210/0014; A61F 2210/0076; A61F 2230/0086; A61F 2250/0004

(57) ABSTRACT

The present technology is directed to adjustable shunting systems for draining fluid from a first body region to a second body region, and can include a shunting element, an aperture in the shunting element, and an actuator for selectively controlling the flow of fluid through the aperture. The actuator can include a gating element moveable between a first position in which it does not substantially interfere with fluid flow through the aperture and a second position in which it at least partially blocks fluid flow through the aperture, and a shape memory actuation element configured to move the gating element from the first position to and/or toward the second position when actuated. The system can further include a friction element configured to frictionally engage the gating element to releasably retain the gating element at and/or proximate the second position following actuation of the shape memory actuation element.

### CROSS-REFERENCE TO RELATED APPLICATIONS

The present application claims priority to U.S. Provisional Patent Applications Nos. 63/175,147, filed Apr. 15, 2021, and 63/216,801, filed Jun. 30, 2021, the disclosures of which are both incorporated by reference herein in their entireties.

### TECHNICAL FIELD

The present technology generally relates to implantable medical devices and, in particular, to shunting systems and associated methods for selectively controlling fluid flow between a first body region and a second body region of a patient.

### BACKGROUND

Implantable shunting systems are widely used to treat a variety of patient conditions by shunting fluid from a first body region/cavity to a second body region/cavity. The flow of fluid through the shunting systems is primarily controlled by the pressure gradient across the shunt and the physical characteristics of the flow path defined through the shunt (e.g., the resistance of the shunt lumen). However, most shunting systems have a single static flow path that is not adjustable. Accordingly, one challenge with conventional shunting systems is selecting the appropriate size shunt for a particular patient. A shunt that is too small may not provide enough therapy to the patient, while a shunt that is too large may create new issues in the patient. Despite this, most conventional shunts cannot be adjusted after implantation and, therefore, cannot be adjusted or titrated to meet the patient's individual and variable needs.

### BRIEF DESCRIPTION OF THE DRAWINGS

Many aspects of the present technology can be better understood with reference to the following drawings. The components in the drawings are not necessarily drawn to scale. Instead, emphasis is placed on illustrating clearly the principles of the present technology. Furthermore, components can be shown as transparent in certain views for clarity of illustration only and not to indicate that the component is necessarily transparent. Components may also be shown schematically.

FIG. 1A illustrates an adjustable shunting system configured in accordance with select embodiments of the present technology.

FIG. 1B is an enlarged view of a plate assembly of the system shown in FIG. 1A and configured in accordance with select embodiments of the present technology.

FIG. 1C is an enlarged view of a portion of the plate assembly shown in FIG. 2 and configured in accordance with select embodiments of the present technology.

FIGS. 2A-2D illustrate an actuator for use with an adjustable shunting system and configured in accordance with select embodiments of the present technology.

FIGS. 3A-3C illustrate another actuator for use with an adjustable shunting system and configured in accordance with select embodiments of the present technology.

FIGS. 4A-4C illustrate another actuator for use with an adjustable shunting system and configured in accordance with select embodiments of the present technology.

FIG. 5 illustrates an actuator for use with an intraocular shunting system and configured in accordance with select embodiments of the present technology.

FIG. 6A illustrates another adjustable shunting system configured in accordance with select embodiments of the present technology.

FIG. 6B is an enlarged view of a portion of the adjustable shunting system shown in FIG. 5A and configured in accordance with select embodiments of the present technology.

### DETAILED DESCRIPTION

The present technology is directed to adjustable shunting systems for draining fluid from a first body region to a second body region of a patient. The adjustable shunting systems can include a shunting element configured to extend between the first body region and the second body region, an aperture fluidly connecting an exterior of the shunting element to an interior of the shunting element, and an actuator for selectively controlling the flow of fluid through the aperture. To do so, the actuator can include (a) a gating element moveable between a first position in which it does not substantially interfere with fluid flow through the aperture and a second position in which it at least partially blocks fluid flow through the aperture, and (b) a shape memory actuation element configured to move the gating element from the first position to and/or toward the second position when actuated. The system can further include a friction element configured to frictionally or otherwise mechanically engage the gating element to releasably retain the gating element at and/or proximate the second position following actuation of the shape memory actuation element. For example, the friction element can engage an end portion of the gating element with a force low enough to permit translation of the end portion during actuation of an actuation element, but high enough to prevent or at least reduce translation of the end portion after the actuation event. For example, in some embodiments (1) a first translational (e.g., rotational) force imparted on the gating element during actuation of the actuation element is greater than the static and dynamic friction between the gating element and the friction element, and (2) a second translational (e.g., rotational) force imparted on the gating element after actuation of the actuation element is less than the static friction between the gating element and the friction element. In some embodiments, the system can also include a sealing element on the gating element to improve a seal at the aperture when the gating element is in the second position.

Shape memory actuators for adjustable shunting systems have been previously described, such as in U.S. patent application Ser. No. 17/175,332, the disclosure of which is incorporated by reference herein in its entirety. Depending on the structure, fabrication process, microstructure, and/or operating conditions of the shape memory actuator, the shape memory actuators may be subject to a recovery effect in which, following an actuation event that imparts a geometric change in the actuator, the actuator moves at least slightly back toward its pre-actuated geometric configuration. For example, in shape memory actuators having two opposing but coupled actuation elements, actuation of a first actuation element moves the first actuation element toward an actuated configuration (e.g., its preferred geometry) but generally deforms a second actuation element relative to its preferred geometry. However, following actuation, the first actuation element may move at least slightly back toward its pre-actuated configuration as it seeks equilibrium with an external (e.g., mechanical) stress pushing it away from its preferred geometry (e.g., stress imparted by the second actuation element being further deformed relative to its preferred geometry). This movement back toward its pre-actuated configuration following actuation is referred to herein as a “recovery motion.” “recovery movement,” “reactive motion.” “recoil,” or “equilibrating movement.” When utilizing a shape memory actuator to move a gating element that controls fluid flow through an aperture, this recovery motion may cause an undesirable motion of the gating element that reduces the level of flow control imparted by the actuator (e.g., by causing a gating element to move away from a desired position following actuation). The present technology is expected to address the effect of recovery motion on gating elements by including features, such as frictional elements, that reduce or eliminate the effect of recovery motion on the gating element, thereby improving control imparted by the actuator. The present technology is also expected to improve fluid control by including sealing elements that can selectively form a partial or complete fluidic seal with the aperture to prevent fluid from flowing through the shunting system.

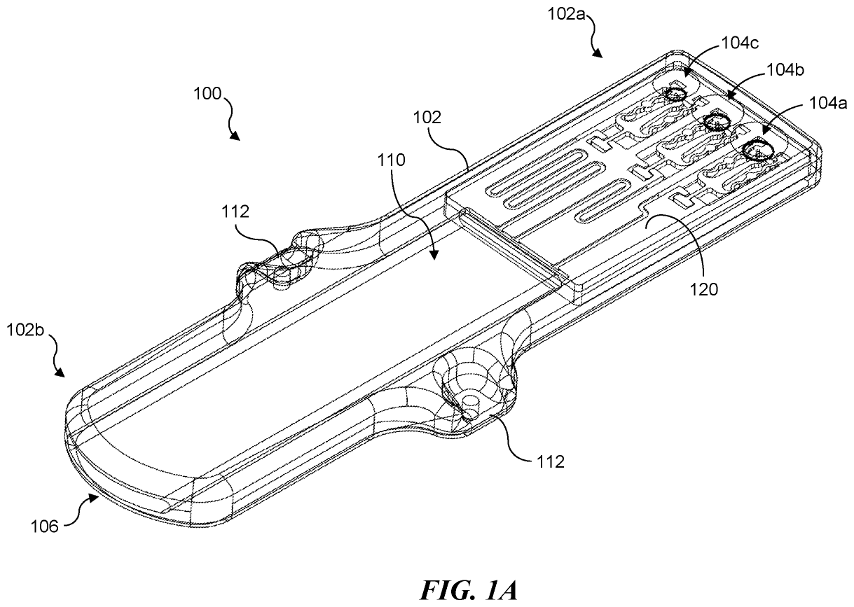

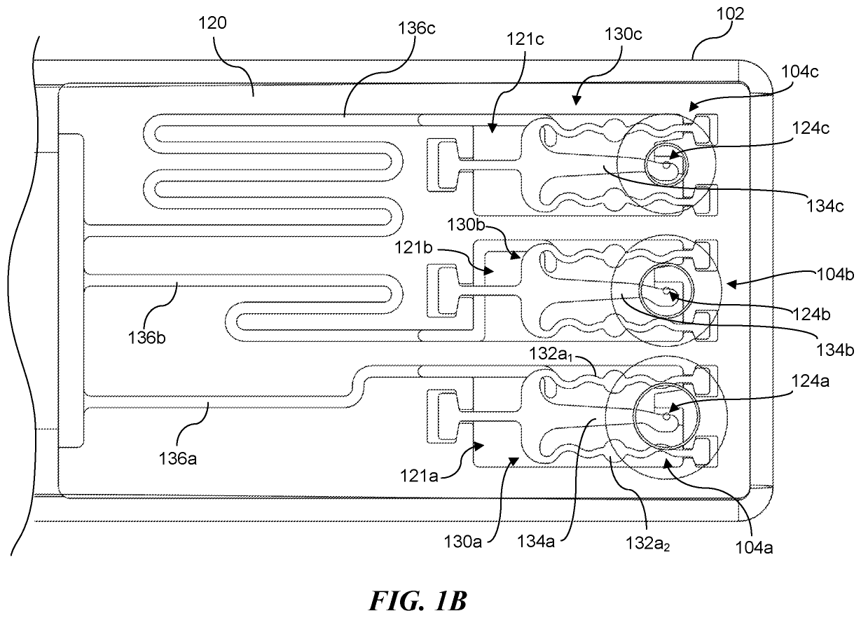

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the examples but are not described in detail with respect to FIGS. 1A-6B.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

Reference throughout this specification to relative terms such as, for example, “generally,” “approximately,” and “about” are used herein to mean the stated value plus or minus 10%. Reference throughout this specification to the term “resistance” refers to fluid resistance unless the context clearly dictates otherwise. The terms “drainage rate,” “flow rate.” and “flow” are used interchangeably to describe the movement of fluid through a structure.

As used herein, the terms “friction element” and “frictional element” are used interchangeably to describe a first structure that is configured to physically contact/engage a second structure. These terms are not limited to configurations in which contact occurs or is configured to occur between parallel surfaces of the first and second structures. Rather, the terms “friction element” and “frictional element” include a first structure that is configured to physically engage/contact a portion of a second structure to at least partially resist motion of the second structure, regardless of the orientation of engagement between the structures.

Although certain embodiments herein are described in terms of shunting fluid from an anterior chamber of an eye, one of skill in the art will appreciate that the present technology can be readily adapted to shunt fluid from and/or between other portions of the eye, or, more generally, from and/or between a first body region and a second body region. Moreover, while the certain embodiments herein are described in the context of glaucoma treatment, any of the embodiments herein, including those referred to as “glaucoma shunts” or “glaucoma devices” may nevertheless be used and/or modified to treat other diseases or conditions, including other diseases or conditions of the eye or other body regions. For example, the systems described herein can be used to treat diseases characterized by increased pressure and/or fluid build-up, including but not limited to heart failure (e.g., heart failure with preserved ejection fraction, heart failure with reduced ejection fraction, etc.), pulmonary failure, renal failure, hydrocephalus, and the like. Moreover, while generally described in terms of shunting aqueous, the systems described herein may be applied equally to shunting other fluid, such as blood or cerebrospinal fluid, between the first body region and the second body region.

FIG. 1A illustrates a shunting system 100 (“the system 100”) configured in accordance with select embodiments of the present technology. As described in greater detail below, the system 100 is configured to provide an adjustable therapy for draining fluid from a first body region, such as to drain aqueous from an anterior chamber of a patient's eye.

The system 100 includes a generally elongated housing 102 and a plate assembly 120. The elongated housing 102 (which can also be referred to as a casing, membrane, shunting element, or the like) extends between a first end portion 102a and a second end portion 102b. The elongated housing 102 substantially and/or fully encases the plate assembly 120 at or proximate the first end portion 102a. In the illustrated embodiment, the elongated housing 102 includes three openings 104 (e.g., a first opening 104a, a second opening 104b, and a third opening 104c) that align with respective fluid inlets in the plate assembly 120, as described in further detail below with respect to FIG. 1B. The elongated housing 102 further includes a main fluid conduit 110 fluidly coupling the plate assembly 120 to one or more fluid outlets 106 positioned proximate the second end portion 102b of the elongated housing 102. In some embodiments, the elongated housing 102 is composed of a slightly elastic or flexible biocompatible material (e.g., silicone, etc.). The elongated housing 102 can also optionally have one or more wings or appendages 112 having holes (e.g., suture holes) for securing the elongated housing 102 in a desired position.

The plate assembly 120 (which can also be referred to as a flow control plate, a flow control cartridge, a plate structure, or the like) is positioned within the elongated housing 102 and is configured to control the flow of fluid through the system 100. For example, as best shown in FIG. 1B, the plate assembly 120 includes one or more fluid apertures or inlets 124 (e.g., a first fluid inlet 124a, a second fluid inlet 124b, and a third fluid inlet 124c) that align with corresponding openings 104a-c in the elongated housing 102. The fluid inlets 124 permit fluid to enter an interior of the plate assembly 120 (and thus an interior of the elongated housing 102) from an environment external to the system 100. In some embodiments, an upper surface of the plate assembly 120 forms a substantial fluid seal with an interior surface of the elongated housing 102 at the first end portion 102a such that the only way for fluid to enter the system 100 is through the fluid inlets 124. Accordingly, for fluid to flow through the system 100, it generally must flow through the plate assembly 120.

The fluid path through the plate assembly 120 depends on which fluid inlet 124 the fluid enters through. For example, fluid that enters the system 100 via the first fluid inlet 124a flows into a first chamber 121a of the plate assembly 120 and drains to the main fluid conduit 110 via a first channel 136a. Fluid that enters the system 100 via the second fluid inlet 124b flows into a second chamber 121b of the plate assembly 120 and drains to the main fluid conduit 110 via a second channel 136b. Fluid that enters the system 100 via the third fluid inlet 124c flows into a third chamber 121c of the plate assembly and drains to the main fluid conduit 110 via a third channel 136c. The chambers 121a-c can be fluidly isolated such that there are three discrete flow paths through the plate assembly 120. The channels 136a-c can also have different geometric configurations (e.g., lengths) relative to one another such that they have different fluid resistances, and thus provide different flow rates for a given pressure. The relative level of therapy provided by each fluid path can be different so that a user may adjust the level of therapy provided by the system 100 by selectively opening and/or closing various fluid paths (e.g., by selectively interfering with or permitting flow through individual fluid inlets 124), as described below. For example, under a given pressure, when fluid enters primarily through the first fluid inlet 124a, the system 100 can provide a first drainage rate, when fluid enters primarily through the second fluid inlet 124b, the system 100 can provide a second drainage rate less than the first drainage rate, and when fluid primarily enters through the third fluid inlet 124c, the system 100 can provide third drainage rate less than the first drainage rate. In other embodiments, the channels 136a-c can have the same or generally the same geometric configurations such that they have the same or generally the same fluid resistances, and thus provide similar flow rates for a given pressure.

The plate assembly 120 is configured to selectively control the flow of fluid entering the system 100. In particular, the plate assembly 120 includes a first actuator 130a positioned in the first chamber 121a and configured to control the flow of fluid through the first fluid inlet 124a, a second actuator 130b positioned in the second chamber 121b and configured to control the flow of fluid through the second fluid inlet 124b, and a third actuator 130c positioned in the third chamber 121c and configured to control the flow of fluid through the third fluid inlet 124c. The first actuator 130a can include a first projection or gating element 134a configured to moveably interface with the first fluid inlet 124a, e.g., to move between a first (e.g., “open”) position in which the gating element 134a does not substantially prevent fluid from flowing through the first fluid inlet 124a (e.g., by not interfering with the first fluid inlet 124a) and a second (e.g., “closed”) position in which the gating element 134a substantially prevents fluid from flowing through the first fluid inlet 124a (e.g., by blocking the first fluid inlet 124a). In some embodiments, the first actuator 130a is designed such that in the second (e.g., “closed”) position, there is a purposeful leak through the first fluid inlet 124a (e.g., the gating element 134a does not completely block fluid flow through the first fluid inlet 124a). In some embodiments, the gating element 134a can be configured to move to one or more intermediate positions between the first (e.g., open) and the second (e.g., closed) position. The second actuator 130b can include a second gating element 134b and the third actuator 130c can include a third gating element 134c that operate in a similar manner as the gating element 134a (e.g., moveable between open and closed positions relative to the second fluid inlet 124b and the third fluid inlet 124c).

The first actuator 130a can further include a first actuation element 132a₁ and a second actuation element 132a₂ that drive movement of the gating element 134a between the first (e.g., open) position and the second (e.g., closed) position. The first actuation element 132a₁ and the second actuation element 132a₂ can be composed at least partially of a shape memory material or alloy (e.g., nitinol). Accordingly, the first actuation element 132a₁ and the second actuation element 132a₂ can be transitionable at least between a first material phase or state (e.g., a martensitic state, a R-phase, a composite state between martensitic and R-phase, etc.) and a second material phase or state (e.g., an austenitic state, an R-phase state, a composite state between austenitic and R-phase, etc.). In the first material state, the first actuation element 132a₁ and the second actuation element 132a₂ may have reduced (e.g., relatively less stiff) mechanical properties that cause the actuation elements to be more easily deformable (e.g., compressible, expandable, etc.) relative to when the actuation elements are in the first material state. In the second material state, the first actuation element 132a₁ and the second actuation element 132a₂ may have increased (e.g., relatively more stiff) mechanical properties relative to the first material state, causing an increased preference toward a specific preferred geometry (e.g., original geometry, manufactured or fabricated geometry, heat set geometry, etc.). The first actuation element 132a₁ and the second actuation element 132a₂ can be selectively and independently transitioned between the first material state and the second material state by applying energy (e.g., laser energy, electrical energy, etc.) to the first actuation element 132a₁ or the second actuation element 132a₂ to heat it above a transition temperature (e.g., above an austenite finish (A_{f}) temperature, which is generally greater than body temperature). If the first actuation element 132a₁ (or the second actuation element 132a₂) is deformed relative to its preferred geometry when heated above the transition temperature, the first actuation element 132a₁ (or the second actuation element 132a₂) will move to and/or toward its preferred geometry. In some embodiments, the first actuation element 132a₁ and the second actuation element 132a₂ are operably coupled such that, when the actuated actuation element (e.g., the first actuation element 132a₁) transitions toward its preferred geometry, the non-actuated actuation element (e.g., the second actuation element 132a₂) is further deformed relative to its preferred geometry.

As the temperature falls back below the transition temperature (e.g., after cessation of energy application), the actuated actuation element (e.g., the first actuation element 132a₁) will transition to and/or toward its first material state and may exhibit some recovery or reactive motion back toward its pre-actuated geometry as it seeks equilibrium with an external (e.g., mechanical) stress pushing the actuation element away from its preferred geometry (e.g., stress imparted by the non-actuated actuation element being further deformed relative to its preferred geometry). Depending on the degree of deformation in the actuation element before actuation and the degree of external stress acting on the actuation element, the recovery motion generally does not cause the actuation element to return all the way to its pre-actuated geometry, but rather to an intermediate geometry between the preferred geometry and the pre-actuated geometry. Because the actuation elements are coupled to the gating element 134a, the recovery motion in the actuation element may also cause a corresponding motion in the gating element 134a, even though minimal and/or no phase transformation occurs there. However, as described below with respect to FIG. 1C, the plate assembly 120 can include one or more features that reduce and/or prevent the recovery motion of the actuation element from causing a corresponding motion of the gating element 134a, and/or that reduce and/or prevent the recovery motion of the actuation element itself.

The first actuation element 132a₁ and the second actuation element 132a₂ generally act in opposition. For example, the first actuation element 132a₁ can be actuated to move the gating element 134a to and/or toward the first (e.g., open) position, and the second actuation element 132a₂ can be actuated to move the gating element 134a to and/or toward the second (e.g., closed) position. Additionally, as described above, the first actuation element 132a₁ and the second actuation element 132a₂ can be coupled such that as one moves toward its preferred geometry upon material phase transition, the other is deformed relative to its preferred geometry. This enables the actuation elements 132a to be repeatedly actuated and the gating element 134a to be repeatedly cycled between the first (e.g., open) position and the second (e.g., closed) position. However, because the first actuation element 132a₁ and the second actuation element 132a₂ are coupled, stress within the first actuation element 132a₁ can cause a recovery motion in the second actuation element 132a₂ following actuation of the second actuation element 132a₂, and stress within the second actuation element 132a₂ can cause a recovery motion in the first actuation element 132a₁ following actuation of the first actuation element 132a₁.

The second actuator 130b and the third actuator 130c can also each include a pair of opposing shape-memory actuators and operate in the same or similar fashion as the first actuator 130a. Additional details regarding the operation of shape memory actuators, as well as adjustable glaucoma shunts, are described in U.S. patent application Ser. No. 17/175,332, U.S. Patent App. Publication No. 2020/0229982, and International Patent Application Nos. PCT/US20/55144, PCT/US20/55141, PCT/US21/14774, PCT/US21/18601, PCT/US21/23238, and PCT/US21/27742, the disclosures of which are incorporated by reference herein in their entireties and for all purposes.

In some embodiments, the plate assembly 120 is formed in part by a plurality of discrete sheets (e.g., glass sheets) bonded together (e.g., via chemical bonding, welding, gluing, or the like). The plurality of discrete sheets can be designed to form the channels 136a-c and the chambers 121a-c. Additional details of, including the manufacturing of, plate assemblies having aspects similar to the plate assembly 120 are described in U.S. Provisional Patent Application No. 63/140,655, the disclosure of which is incorporated by reference herein in its entirety.

FIG. 1C is an enlarged view of a portion of the plate assembly 120 illustrating the first chamber 121a with the first actuator 130a omitted for clarity. As illustrated, the plate assembly 120 can include a stopping element 127a (e.g., wall) in the first chamber 121a that engages the gating element 134a (FIG. 1B) as the gating element 134a moves from the first (e.g., open) position to and/or toward the second (e.g., closed) position. This prevents the gating element 134a from moving past the second (e.g., closed) position when the second actuation element 132a₂ (FIG. 1B) is actuated.

The plate assembly 120 also includes a raised portion or ledge 125a positioned generally beneath the first fluid inlet 124a (e.g., positioned such that an axis extending through the center of the first fluid inlet 124a also extends through the ledge 125a). The ledge 125a is raised relative to the portion of the plate assembly that forms a “floor” of the first chamber 121a. While the ledge 125a is shown in FIG. 1C with a flat step cross-sectional profile, the ledge cross-sectional profile may be any shape that is raised relative to the floor including triangular, circular, sinusoidal, parabolic, hyperbolic, spherical, conical, etc. The ledge 125a can serve a number of purposes expected to improve operation of the first actuator 130a, described below.

The ledge 125a can reduce undesirable motion of the gating element 134a (e.g., by reducing the effects of recovery motion on the gating element 134a) as it moves from the first (e.g., open) position to and/or toward the second (e.g., closed) position. As previously described with reference to FIG. 1B, if the gating element 134a is in a first (e.g., open) position, actuating the second actuation element 132a₂ moves the gating element 134a toward the second (e.g., closed) position (e.g., such that it is positioned generally below the first fluid inlet 124a). However, once heat is removed from the second actuation element 132a₂, it may exhibit a recovery motion as it transitions back toward its first material state. Because the second actuation element 132a₂ is connected to the gating element 134a, this recovery motion can also cause a corresponding (and undesirable) motion in the gating element 134a back toward the first (e.g., open) position, thereby moving the gating element 134a away from the desired stopping position, which may partially or fully unblock the first fluid inlet 124a. This is disadvantageous because it may allow fluid to enter and/or may increase the amount of fluid entering the plate assembly 120 via the first fluid inlet 124a even through a user desired to “close” the first fluid inlet 124a. To prevent or reduce this undesirable motion of the gating element 134a, the ledge 125a is configured to form a frictional interface with a first (e.g., lower) surface of the gating element 134a when the gating element 134a is in the second (e.g., closed) position. Accordingly, the ledge 125a can also be referred to as a “friction element.” The force of the frictional interface can be weak enough to allow the gating element 134a to move in the selected actuation direction, while being strong enough to prevent or at least hinder the gating element 134a from moving back toward the open position if the first actuator 130a exhibits a recovery motion. Thus, the ledge 125a helps to “hold” the gating element 134a in the closed position. The force of the frictional interface is weak enough that it can be overcome by actuating the first actuation element 132a₁ to purposefully drive the gating element 134a back to the open position if a user desires to “open” the first fluid inlet 124a.

The ledge 125a can also improve the seal between the gating element 134a and the first fluid inlet 124a when the gating element 134a is in the closed position. For example, the ledge 125a may direct the gating element 134a upwardly toward the first fluid inlet 124a such that a second (e.g., upper) surface of the gating element contacts the lower side of the first fluid inlet 124a. This contact can prevent or at least substantially prevent fluid from flowing into the plate assembly 120 via the first fluid inlet 124a. Of course, there may still be some small leak even when the gating element 134a is in the closed position. However, incorporation of the ledge 125a is expected to advantageously reduce any leak. The ledge 125a may also prevent the gating element 134a from deflecting downwardly and away from the first fluid inlet 124a in response to pressure increases in the environment external to the first fluid inlet 124a.

The ledge 125a can therefore be configured to (1) form a frictional interface with the lower surface of the gating element 134a, and (2) improve the contact (and therefore the seal) between the upper surface of the gating element 134a and the first fluid inlet 124a. In some embodiments, the ledge 125a is a plateau-like structure, in which a plane of the ledge is generally parallel to a plane of the lower surface of the first chamber 121a. In other embodiments, the ledge 125a can be wedge-shaped structure that forms a ramp having a non-parallel surface with the plane of the lower surface of the first chamber 121a. In some embodiments, the ledge 125a is textured or has other surface features to improve the frictional interference with the gating element 134a.

In some embodiments, the ledge 125a is formed on one of the plurality of sheets forming the plate assembly 120. For example, the ledge 125a can be integral with the sheet that forms the lower surface of the first chamber 121a. Accordingly, the ledge 125a can be composed of the same material as the sheets (e.g., glass). In other embodiments, the ledge 125a can be a separate feature that is adhered to (e.g., welded, glued, or otherwise secured to) the lower surface of the first chamber 121a.

In some embodiments, the ledge 125a is a first ledge, and the first chamber 121a further includes a second ledge (not shown). The second ledge can also be positioned on the surface of the plate assembly that forms the “floor” of the first chamber 121a. However, unlike the ledge 125a, the second ledge can be positioned such that the gating element 134a frictionally engages the second ledge when it is moved to the open position. The second ledge can therefore prevent undesirable movement of the gating element 134a back toward the closed position following actuation of the first actuation element 132a₁ to move the gating element 134a from the closed position to the open position.

Although not described for brevity, the plate assembly 120 can also one or more ledges (not shown) in the second chamber 121b for reducing the effects of recovery motion during actuation of the second actuator 130b and one or more ledges (not shown) in the third chamber 121c for reducing the effects of recovery motion during actuation of the third actuator 130c. These additional ledges can operate in a manner substantially similar to the ledge 125a described herein. Moreover, although described as having three inlets 124 and three actuators 130, the plate assembly 120 can have more or fewer inlets 124 and actuators 130. For example, the plate assembly 120 can have one, two, four, five, six, or more inlets 124 and, one, two, four, five, six, or more actuators 130.

The system 100 can be used to treat a number of patient conditions. For example, the system 100 can be used to drain aqueous from the anterior chamber of the eye to treat glaucoma. Accordingly, when the system 100 is implanted in an eye to treat glaucoma, the first end portion 102a of the elongated housing 102 can be positioned within an anterior chamber of the patient's eye such that the fluid inlets 124 are in fluid communication with the anterior chamber, and the second end portion 102b can be positioned in a target outflow location, such as a subconjunctival bleb space, such that the fluid outlet 106 is in fluid communication with the target outflow location. Aqueous can flow into the elongated housing via the fluid inlets 124, through the plate assembly 120, into the main fluid conduit 110, and exit via the fluid outlet 106.

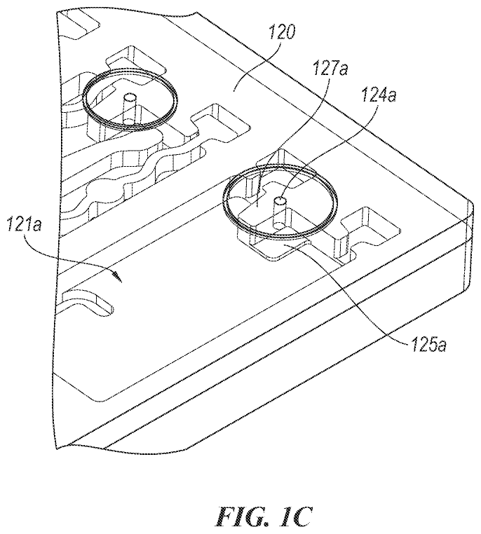

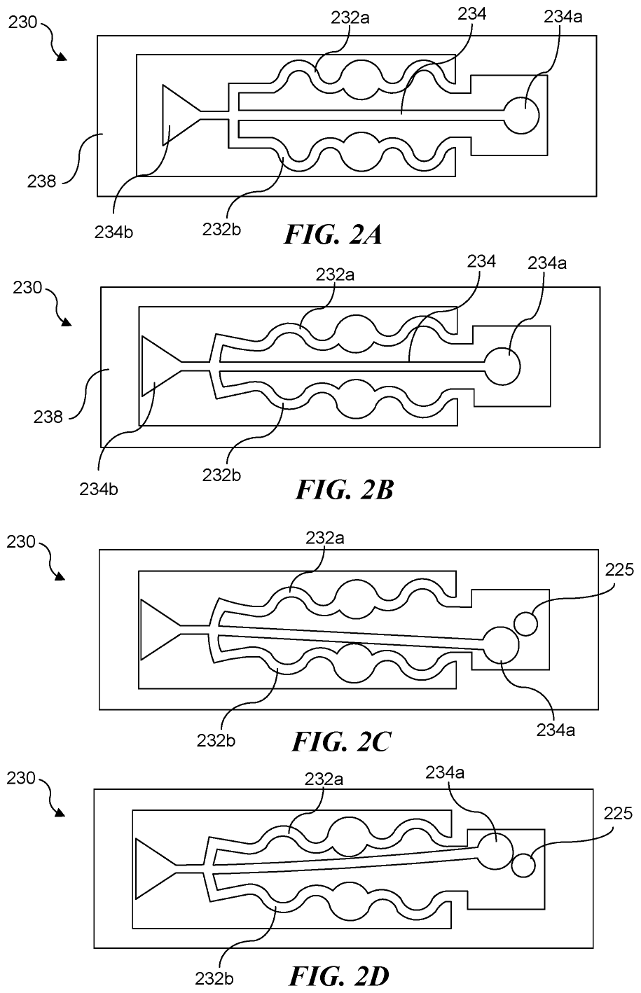

In addition to or in lieu of the ledge 125 described above, the systems described herein can include other features that reduce or otherwise inhibit undesirable motion of the gating element caused by a recovery motion in an actuation element following actuation of the actuation element. For example, FIGS. 2A-2D illustrate an actuator 230 for use with an adjustable shunting system (e.g., the system 100 of FIGS. 1A-1C) and configured in accordance with select embodiments of the present technology. The actuator 230 can be generally similar to the actuators 130 described above with reference to FIGS. 1A-IC. For example, referring generally to FIGS. 2A-2D, the actuator 230 can include a gating element 234 positioned between a first actuation element 232a and a second actuation element 232b. The gating element 234 can include a first (e.g., distal) end portion 234a configured to interface with a corresponding fluid inlet or aperture of a shunting system (e.g., fluid inlet 124 of the system 100; not shown in FIGS. 2A-2D), and a second (e.g., proximal) end portion 234b opposite the first end portion 234a. The first actuation element 232a can be configured to move the first end portion 234a of the gating element 234 in a first direction to and/or toward a first (e.g., open) position (e.g., to unblock the fluid inlet 124 shown in FIGS. 1A-1C), and the second actuation element 232b can be configured to move the first end portion 234a of the gating element 234 in a second direction to and/or toward a second (e.g., closed) position (e.g., to interfere with the fluid inlet 124 shown in FIGS. 1A-IC). The actuator 230 can optionally include an outer perimeter 238 that surrounds or at least partially surrounds the gating element 234, the first actuation element 232a, and the second actuation element 232b.

FIG. 2A illustrates the actuator 230 in an unstrained configuration (e.g., the “as-manufactured” or “as-cut” configuration). In the unstrained configuration, the first actuation element 232a and the second actuation element 232b both occupy their preferred geometries. FIG. 2B illustrates the actuator 230 in a strained configuration (e.g., the “loaded” or “tensioned” configuration). In the strained configuration, both the first actuation element 232a and the second actuation element 232b are deformed (e.g., lengthened, tensioned, etc.) relative to their preferred geometries, causing stress in both the first actuation element 232a and the second actuation element 232b. In the illustrated embodiment, the actuator 230 can be transitioned between the unstrained configuration and the strained configuration by securing the second end portion 234b of the gating element 234 to the perimeter 238, as shown in FIG. 2B. However, in other embodiments the actuator 230 can be transitioned to the strained configuration by otherwise deforming and securing the first and second actuation elements. In some embodiments, the actuator 230 can be generally similar to the shape memory actuators described in U.S. patent application Ser. No. 17/175,332, previously incorporated by reference herein.

FIGS. 2C and 2D illustrate operation of the actuator 230. In addition to the actuator 230, FIGS. 2C and 2D also illustrate a frictional element 225 (which can also be referred to as a latching element or a retention element). Similar to the ledge 125 described with respect to FIG. 1C, the frictional element 225 can be formed on and/or coupled to one of the plurality of sheets forming the plate assembly that houses the actuator 230 (e.g., the plate assembly 120, shown in FIGS. 1A-IC). The frictional element 225 can also be secured to another suitable portion of the shunting system. The frictional element 225 can be a pin, tab, nub, bump, projection, or the like, and can have any suitable shape/geometry (e.g., cylindrical, cuboid, rectangular, pyramidal, etc.). In some embodiments, the frictional element 225 has a height equal to or greater than a thickness of the actuator 230).

As shown in FIGS. 2C and 2D, the frictional element 225 is configured to interfere with and/or engage (e.g., physically, mechanically, frictionally, etc.) the first end portion 234a of the gating element 234. In this way, and as described in more detail below, the frictional element 225 can bias the first end portion 234a of the gating element 234 toward a specific position and/or inhibit movement of the first end portion 234a away from a specific position (e.g., via static friction). For example, when the first end portion 234a is in the first (e.g., open position) as shown in FIG. 2C, the friction element 225 can bias the first end portion 234a of the gating element 234 toward and/or hold/retain the first end portion 234a at the first position. When the first end portion 234a is in the second (e.g., closed position) as shown in FIG. 2D, the frictional element 225 can bias the first end portion 234a of the gating element 234 toward and/or hold/retain the first end portion 234a at second position. Thus, the frictional element 225 generally at least partially resists movement of the first end portion 234a away from the position it occupies at any given time.

The biasing force (e.g., the static friction) created by the engagement of the gating element 234 and the frictional element 225 can be overcome by actuating one of the actuation elements 232. For example, to move the gating element 234 from the first (e.g., open) position shown in FIG. 2C to the second (e.g., closed) position shown in FIG. 2D, the second actuation element 232b can be actuated, such as by heating the second actuation element 232b above its transition temperature. As the second actuation element 232b transitions from the first material state (e.g., martensitic) to the second material state (e.g., austenitic), the increased mechanical properties (e.g., increased stiffness) within the second actuation element 232b transition the second actuation element 232b toward its preferred geometry, causing the second actuation element 232b to contract. The contraction of the second actuation element 232b imparts a first rotational force on the gating element 234 toward the second (e.g., closed) position. This first rotational force is initially resisted by the static friction between the frictional element 225 and the first end portion 234a of the gating element 234. However, as the first rotational force increases as the second actuation element 232b contracts further toward its preferred geometry, the first rotational force eventually overcomes (e.g., becomes greater than) the frictional or other mechanical biasing force between the frictional element 225 and the first end portion 234a of the gating element 234. For example, the first rotational force can be greater than the static and dynamic frictional force between the gating element 234 and the friction element 225. As a result, the first end portion 234a “slips” or “slides” past the frictional element 225 to occupy the second (e.g., closed) position, as shown in FIG. 2D (e.g., similar to a pawl sliding over a tooth of a ratchet). In some embodiments, therefore, the actuator 230 demonstrates hysteresis as a result of the frictional element 225.

After energy delivery terminates and the second actuation element 232b cools, it may exhibit a recovery motion as it transitions back toward its first material state as it seeks equilibrium with an external (e.g., mechanical) stress pushing the second actuation element 232b away from its preferred geometry (e.g., stress imparted by the first actuation element 232a being further deformed relative to its preferred geometry). Because the second actuation element 232b is connected to the gating element 234, this recovery motion can also cause a second rotational force against the gating element 234 back toward the first (e.g., open) position (e.g., opposite the first rotational force). However, once the gating element 234 is in the second (e.g., closed) position, the frictional element 225 imparts a frictional or other mechanical biasing force against the first end portion 234a of the gating element 234 that is greater than the second rotational force caused by recovery motion. For example, the second rotational force is less than the static frictional force between the gating element 234 and the frictional element 225. Thus, the frictional element 225 retains the first end portion 234a in the second (e.g., closed) position shown in FIG. 2D, even as the second actuation element 232b transitions back to the first material state (thereby returning to a similar stiffness as the first actuation element 232a) and exhibits a recovery motion.

Accordingly, the frictional element 225 is expected to help reduce or mitigate the effects of recovery motion on the ability to selectively titrate fluid flow using the actuator 230, similar to the ledge 125 described with respect to FIG. 1C. However, relative to the ledge 125, the frictional element 225 helps reduce and/or mitigate the effects of recovery motion following actuation of both the first actuation element 232a and the second actuation element 232b. For example, the operation described above with respect to FIGS. 2C and 2D can be reversed by delivering energy to heat the first actuation element 232a to move the gating element 234 from the second (e.g., closed) position shown in FIG. 2C to the first (e.g., open) position shown in FIG. 2D. During such operation, the frictional element 225 helps retain the gating element 234 in the first (e.g., open) position following actuation of the first actuation element 232a. The frictional element 225 is therefore expected to increase the consistency of motion in the gating element 234 (e.g., by decreasing the effect of any recovery motion on the gating element 234), and thus increase the fluid control attainable using the actuator 230.

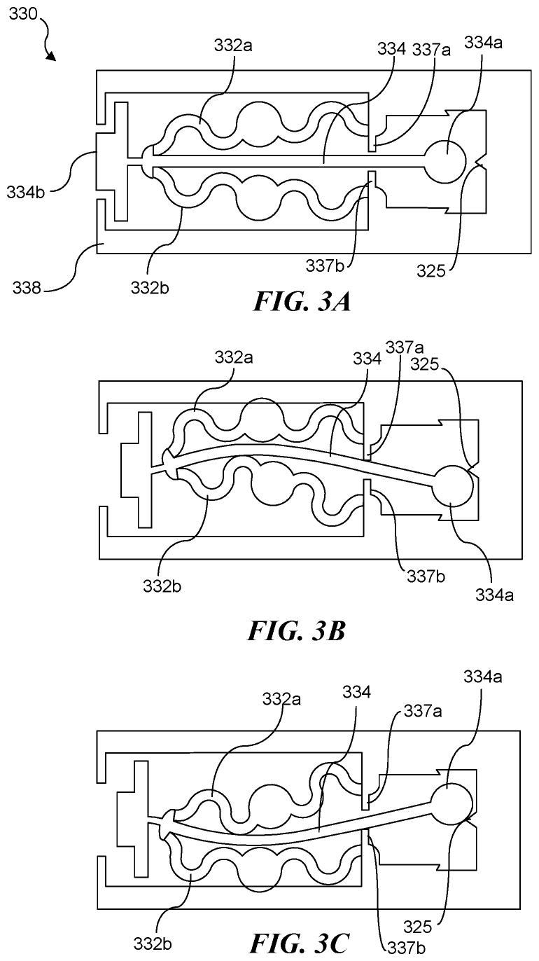

FIGS. 3A-3C illustrate another actuator 330 for use with an adjustable shunting system (e.g., the system 100 of FIGS. 1A-IC) and configured in accordance with select embodiments of the present technology. Referring generally to FIGS. 3A-3C, the actuator 330 can be activated in a generally similar manner to the actuators 130 and 230 described above. For example, the actuator 330 can include a gating element 334 having a first end portion 334a configured to control fluid flow through an inlet (not shown) in the shunting system and a second end portion 334b. The actuator 330 can also include a first actuation element 332a, a second actuation element 332b, and an outer perimeter 338.

FIG. 3A illustrates the actuator 330 in an unstrained (e.g., the “as-manufactured” or “as-cut” configuration) in which a first actuation element 332a and a second actuation element 332b assume their preferred geometries. FIG. 3B illustrates the actuator 330 in a first strained configuration in which at least the second actuation element 332b is deformed (e.g., compressed) relative to its preferred geometry and the gating element 334 is in a first (e.g., open) position in which it does not interfere with the corresponding fluid inlet (not shown). FIG. 3C illustrates the actuator 330 in a second strained configuration in which at least the first actuation element 332a is deformed (e.g., compressed) relative to its preferred geometry and the gating element 334 is in a second (e.g., closed) position configured to interface with the corresponding fluid inlet (not shown). Accordingly, relative to the actuator 230, the actuation elements 332 are configured to be compressed relative to their preferred geometries when in the strained configuration.

The outer perimeter 338 of the actuator 330 can include a first finger 337a and a second finger 337b (collectively referred to as the fingers 337). The first finger 337a and the second finger 337b can form pivot points that engage an intermediate portion of the gating element 334 between the first end portion 334a and the second end portion 334b during actuation of the first actuation element 332a and the second actuation element 332b, respectively. As a result, actuating the first actuation element 332a causes the gating element 334 to pivot or otherwise bend about the first finger 337a, which causes the first end portion 334a to move from the second (e.g., closed) configuration shown in FIG. 3C to and/or toward the first (e.g., open) position shown in FIG. 3B. Actuating the second actuation element 332b causes the gating element 334 to pivot or otherwise bend about the second finger 337b, which causes the first end portion 334a to move from the first (e.g., open) position shown in FIG. 3B to and/or toward the second (e.g., closed) position shown in FIG. 3A. Without being bound by theory, use of the fingers 337 as pivot points may permit the gating element 334 to have a shorter length while still achieving a range of motion sufficient to toggle the first end portion 334a between the first and second positions.

The actuator 330 also includes a frictional element 325 configured to reduce and/or mitigate the effects of recovery motion in the actuator 330 following actuation of the first actuation element 332a and following actuation of the second actuation element 332b. The frictional element 325 can operate in a similar manner as the frictional element 225 described with respect to FIGS. 2C and 2D. For example, the frictional element 225 can engage the first end portion 334a of the gating element 334 with a force low enough to permit translation of the first end portion 334a during actuation of an actuation element, but high enough to prevent or at least reduce translation of the first end portion 334a after the actuation event. Relative to the frictional element 225, however, the frictional element 325 is integral with the actuator 330, and can therefore be fabricated with the other components of the actuator.

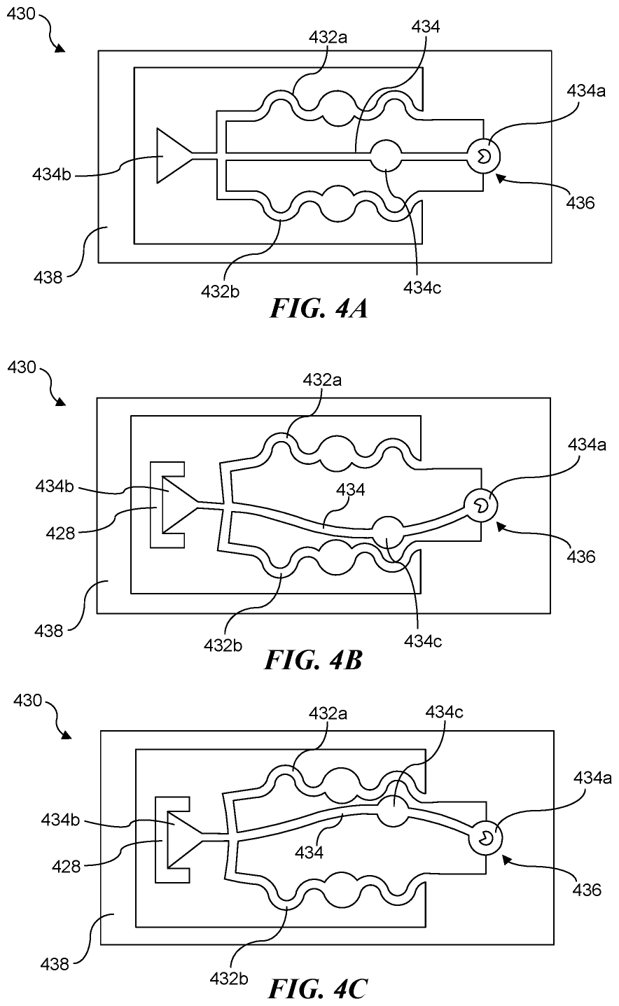

In some embodiments, the actuators can be configured to reduce or otherwise inhibit undesirable motion of the gating element caused by a recovery motion in an actuation element without the use of a frictional element. For example, FIGS. 4A-4C illustrate an actuator 430 for use with an adjustable shunting system (e.g., the system 100 of FIGS. 1A-1C) and configured in accordance with select embodiments of the present technology. The actuator 430 can include certain features generally similar to the actuators 130, 230, and 330 described previously. For example, the actuator 430 can include a gating element 434 having a first end portion 434a and a second end portion 434b, a first actuation element 432a, a second actuation element 432b, and an outer perimeter 438. However, relative to the actuators 230 and 330) described previously, the first end portion 434a of the gating element is not configured to control fluid flow through an inlet (not shown) in the shunting system. Rather the first end portion 434a is coupled to the perimeter 438 at a receiving feature 436 (e.g., groove, notch, etc.) in the perimeter 438, and the gating element 434 further includes an intermediate portion 434c configured to control fluid flow through the inlet (not shown) in the shunting system.

FIG. 4A illustrates the actuator 430 in an unstrained (e.g., the “as-manufactured” or “as-cut” configuration) in which a first actuation element 432a and a second actuation element 432b assume their preferred geometries. FIG. 4B illustrates the actuator 430 in a strained configuration in which at least the first actuation element 432a is deformed (e.g., compressed) relative to its preferred geometry and the gating element 434 is in a first (e.g., open) position in which it does not interfere with the corresponding fluid inlet (not shown). FIG. 4C illustrates the actuator 430 in a strained configuration in which at least the second actuation element 432b is deformed (e.g., compressed) relative to its preferred geometry and the gating element 434 is in a second (e.g., closed) position configured to interface with the corresponding fluid inlet (not shown). The first and second positions of the gating element 434 represent relatively low-internal energy states (e.g., states having relatively low stored energy in the gating element 434), and can therefore be referred to herein as a “first equilibrium position or state” and a “second equilibrium position or state,” respectively. As set forth in detail below, the gating element 434 preferentially exists in (e.g., is biased toward) either the first position or the second position.

The actuator 430) can be transitioned between the unstrained configuration shown in FIG. 4A and the strained configurations shown in FIGS. 4B and 4C by placing the second end portion 434b within a retaining element 428 that helps hold the actuator 430 in the strained (e.g., compressed) configuration. The retention element 428 can be formed on and/or coupled to one of the plurality of sheets forming the plate assembly that houses the actuator 430 (e.g., the plate assembly 120, shown in FIGS. 1A-1C). The retention element 428 can also be secured to another suitable portion of the shunting system. As shown in FIG. 4B, moving the actuator 430 from the unstrained configuration to the strained configuration causes the gating element 434 to bow outwardly relative to its longitudinal axis. In some embodiments, the gating element 434 bows outwardly because the distance between the retention element 428 (which engages the second end portion 434b) and the receiving feature 436 (which engages the first end portion 434a) is less than a length of the gating element 434. Thus, the bowed configurations shown in FIGS. 4B and 4C depict the low-energy states for the gating element 434.

In operation, the gating element 434 can be selectively transitioned between the first (e.g., open) position shown in FIG. 4B and the second (e.g., closed) position shown in FIG. 4C by actuating the first or second actuation element 432. For example, to transition the gating element 434 from the open position to the closed position, the first actuation element 432a can be actuated, such as by heating the first actuation element 432a above its transition temperature. As the first actuation element 432a transitions from the first material state (e.g., martensitic) to the second material state (e.g., austenitic), the increased mechanical properties (e.g., increased stiffness) within the first actuation element 432a transition the first actuation element 432a toward its preferred geometry, causing the first actuation element 432a to lengthen. The lengthening of the first actuation element 432a imparts a first rotational force on the gating element 434 toward the second (e.g., closed) position, as previously described with respect to the actuators 230) and 330.

This first rotational force is initially resisted by the gating element 434. As previously described, the gating element 434 exists in a first equilibrium or low energy state when in the first position shown in FIG. 4B. In some embodiments, the gating element 434 therefore generally has a relatively lower internal stress when in the first position, as compared to other potential configurations of the gating element 434. However, moving the gating element 434 from the open position (e.g., the first low energy state) toward the closed position (e.g., the second low energy state) requires transitioning the gating element 434 through an intermediate position (not shown) in which it at least transiently exists in a higher energy transition state generally having a relatively higher internal stress, as compared to the low internal stress in the low energy states shown in FIGS. 4B and 4C. For example, in some embodiments, the gating element 434 transiently assumes/passes through an s-shaped or other high energy configuration in the intermediate position/higher energy transition state, although other configurations are possible. Thus, to move the gating element 434a from the first position shown in FIG. 4B to the second position shown in FIG. 4C, the gating element 434 must at least transiently pass through a relatively high energy (and high stress) state (referred to herein as moving through an “energy gradient” or “stress gradient”). This energy gradient therefore at least initially resists motion of the gating element 434 from the open position toward the closed position, and therefore at least initially resists movement of the gating element 434 in response to the first rotation force imparted by actuation of the first actuation element 432a. However, as the first actuation element 434a further lengthens toward its preferred geometry, the first rotational force overcomes the biasing force of the energy gradient and the gating element 434 therefore moves from the first position in which it exists in the first low energy state having the relatively low internal stress, through the intermediate position in which it at least transiently exists in the relatively higher energy state having the relatively high internal stress, and to the second position in which it exists in a second low energy state once again having a relatively low internal stress. Of note, the gating element 434 consistently and quickly moves between the first position and the second position upon actuation because the first and second positions represent the low energy states that the gating element 434 is biased toward. Accordingly, the gating element 434 quickly passes through any intermediate high-energy states between the first and second positions. Thus, the gating element 434 can be described herein as “snapping” between the first and second positions upon actuation of the actuator, as the open and closed positions both represent the lowest energy states for the gating element 434.

After energy delivery terminates and the first actuation element 432a cools, it may exhibit a recovery motion as it transitions back toward its first material state, as previously described. For example, the first actuation element 432a may seek equilibrium with an external (e.g., mechanical) stress pushing the first actuation element 432a away from its preferred geometry (e.g., stress imparted by the second actuation element 432b being further deformed relative to its preferred geometry). Because the first actuation element 432a is connected to the gating element 434, this recovery motion can also cause a second rotational force against the gating element 434 back toward the first (e.g., open) position (e.g., opposite the first rotational force). However, the energy gradient biasing the gating element 434 toward the second position prevents or reduces any undesirable motion of the gating element 434 away from the second position during recovery motion of the first actuation element 434a (e.g., because the biasing force created by the energy gradient is greater than the second rotational force).

Accordingly, the actuator 430 is designed to help reduce or mitigate the effects of recovery motion on the ability to selectively titrate fluid flow using the actuator 430. More specifically, this is accomplished by designing the actuator 430 such that the gating element 434 moves through a relatively high energy, high stress intermediate configuration when moving between a first relatively low energy, low stress configuration (e.g., the first position) and a second relatively low energy, low stress configuration (e.g., the second position). Of note, this can be accomplished without requiring an external frictional element acting on the gating element 434. Such configuration is also expected to improve the consistency and repeatability of moving the gating element 434 between the first and second positions (e.g., by minimizing the amount of time the gating element occupies other positions), since the first and second positions represent the two “stable” (e.g., low energy) positions the gating element 434 can occupy.

Although described in terms of transitioning from a first low energy state, through a relatively high energy intermediate state, and to a second low energy state, operation of the actuator 430 can be described in other terms. For example, the gating element may have a first internal stress and/or strain distribution or field when in the first low energy state, a second internal stress and/or strain distribution when in the second low energy state, and a third internal stress and/or strain distribution when in the intermediate high energy state. In such embodiments, the actuator 430 can biased toward the first and second stress distributions (e.g., by virtue of these distributions representing the lowest energy states).

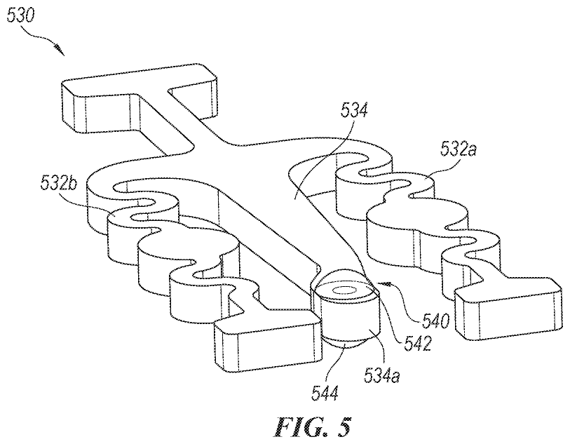

The actuators described herein can include additional features that improve the functioning of the shunting systems. For example, FIG. 5 illustrates an actuator 530 for use with an adjustable shunting system (e.g., the system 100 of FIGS. 1A-IC) and configured in accordance with select embodiments of the present technology. The actuator 530 can be generally similar to the actuators 130, 230, 330, and 430 described with reference to FIGS. 1A-4C. For example, the actuator 530 can include a gating element 534 configured to interface with a corresponding fluid inlet or aperture of a shunting system (e.g., the fluid inlet 124 of the system 100), a first actuation element 532a for moving the gating element 534 in a first direction to and/or toward a first (e.g., open) position (e.g., to unblock the fluid inlet 124), and a second actuation element 532b for moving the gating element 534 in a second direction generally opposite the first direction to and/or a toward a second (e.g., closed) position (e.g., to block the fluid inlet 124).

Relative to the actuators shown in FIGS. 1A-4C, the actuator 530 further includes a sealing element 540) coupled to a first end region 534a of the gating element 534. The sealing element 540) includes a first portion 542 positioned on a first (e.g., upper) surface of the first end region 534a and, in at least some embodiments, optionally includes a second portion 544 positioned on a second (e.g., lower) surface of the first end region 534a. In some embodiments, the first portion 542 and the second portion 544 are connected by a medial portion (not shown) that extends through an aperture in the first end region 534a to thereby secure the sealing element 540 to the first end region 534a. The sealing element 540 can also be secured to the gating element 534 via other suitable techniques, such as mechanical tethers, bands, barbs, staples, sutures, or the like. In some embodiments, the sealing element 540) is a coating or other material on the surface of the gating element 534 (e.g., completely or at least partially surrounding the first end region 534a) and therefore does not necessarily extend through the aperture in the first end region 534a.

In some embodiments, the sealing element 540 is expected to improve the flow-blocking effect of the gating element 534 by increasing the fluid resistance through the fluid inlet 124 (FIG. 1B) when the gating element 534 is in the second (e.g., closed) position. For example, the sealing element 540) can improve the seal between the first end portion 534a of the gating element 534 and the fluid inlet 124 when the gating element 534 is in the second position and help inhibit, reduce, or otherwise control leaks through the fluid inlet 124. In particular, when the gating element 534 is in the second position, the first portion 542 of the sealing element 540) can abut, conform to, and/or be partially inserted into the fluid inlet 124 to reduce and/or eliminate the flow of fluid through the fluid inlet 124. In some embodiments, the sealing element 540) does not eliminate leaks through the fluid inlet 124, but rather provides a consistent fluid resistance through the fluid inlet 124 when the gating element 534 is in the second (e.g., closed) position to provide a known leak. In embodiments in which the sealing element 540) includes the second portion 544 positioned underneath the first end region 534a, the second portion 544 can further direct the gating element 534 toward the fluid inlet 124 by contacting a surface beneath the gating element 534 (e.g., the ledge 125a shown in FIG. 1C).

To further improve its sealant effect, the sealing element 540 can be at least partially composed of an impermeable and partially compressible or flexible material (e.g., an elastomeric material, a material having a low durometer, etc.) that can at least partially conform to the fluid inlet 124 when the gating element 534 is in the second position. For example, the sealing element 540) can be composed of a hydrophobic material such as silicone. In another example, the sealing element 540 can be composed of a hydrophilic material, or a combination of hydrophobic and hydrophilic materials. In some embodiments, the sealing element 540) or at least a portion thereof is composed of a lubricious material, coating, or gel. For example, in some embodiments the first portion 542 of the sealing element 540 is composed of a lubricious material or includes a lubricious coating, and the second portion 544 is composed of a non-lubricious material or coating. As another example, the entire sealing element 540) (including the first portion 542 and the second portion 544) can be composed of a lubricious material and/or include a lubricious coating.

In addition to improving the seal between the gating element 534 and the fluid inlet 124, in some embodiments the sealing element 540) can also increase the frictional interference that holds the gating element 534 at or proximate the second (e.g., closed) position. For example, the contact between the first portion 542 of the sealing element 540 and the surface defining the fluid inlet 124 can cause a frictional force that, in conjunction with the ledge 125a (shown in FIG. 1C) or friction feature 225, 325 (shown in FIGS. 2C-3C) may help prevent or reduce undesirable motion of the gating element 534 following actuation of the actuator 530. Likewise, the contact between the second portion 544 of the sealing element 540 and the surface beneath the actuator 530) can cause a frictional force that further helps prevent or reduce undesirable motion of the gating element 534 following actuation of the actuator 530. In other embodiments, the sealing element 540) does not substantially contribute to the frictional forces that hold the gating element 534 at or proximate the second position (e.g., embodiments in which the sealing element is a hydrophilic and lubricious coating or gel). Although described as being coupled to the gating element 534, in other embodiments the systems described herein can include sealing elements coupled to the surface defining the fluid inlet in lieu of, or in addition to, a sealing element coupled to the gating element. For example, some systems configured in accordance with the present technology could include an impermeable and flexible membrane positioned adjacent the fluid inlet such that, when the gating element is in the first (e.g., open) position, the gating element is at least partially spaced apart from the fluid inlet and therefore permits fluid to enter the system. When the gating element is in the second (e.g., closed) position, the gating element can contact the membrane and press it against and/or into the fluid inlet to form a fluid seal at the fluid inlet. Accordingly, the present technology is not limited to the express embodiments depicted and described herein, and instead encompasses embodiments related to those described herein.

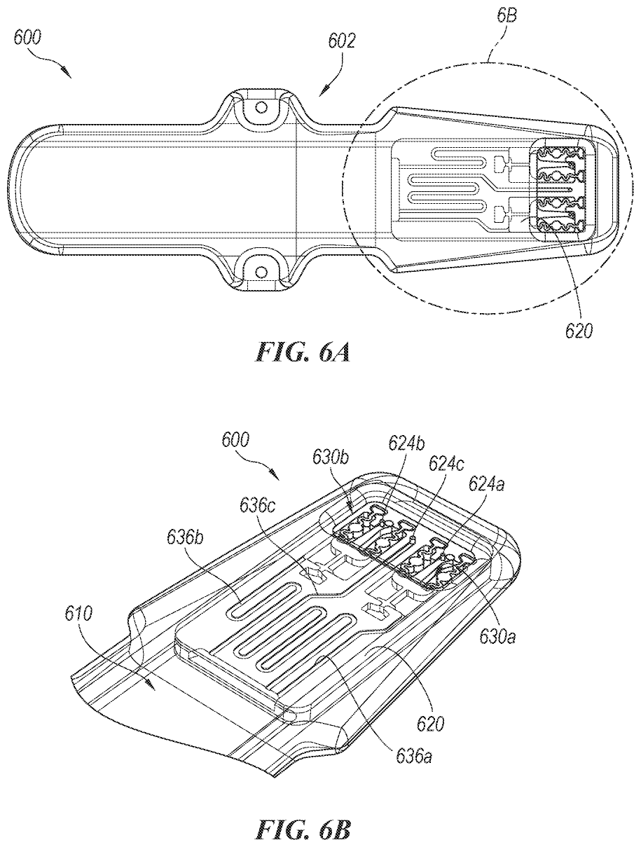

FIGS. 6A and 6B illustrate another shunting system 600 (“the system 600”) configured in accordance with select embodiments of the present technology. The system 600 can be generally similar to the system 100. For example, the system 600 can include a generally elongated housing 602 and a plate assembly 620 configured to provide an adjustable therapy for draining fluid from a first body region, such as to drain aqueous from an anterior chamber of a patient's eye.

As best shown in FIG. 6B, and similar to the system 100, the system 600 includes three flow paths for fluid to drain through the plate assembly 620 and into a main fluid conduit 310 of the elongated housing 602. For example, the plate assembly 620 includes a first fluid inlet 624a, a second fluid inlet 624b, and a third fluid inlet 624c. The first fluid inlet 624a is fluidly coupled to a first channel 636a, the second fluid inlet 624b is fluidly coupled to a second channel 636b, and the third fluid inlet 624c is fluidly coupled to a third fluid channel 636c. Similar to the system 100, the plate assembly 620 includes a first actuator 630a configured to selectively control the flow of fluid through the first fluid inlet 624a and a second actuator 630b configured to selectively control the flow of fluid through the second fluid inlet 624b.

Unlike the system 100, the plate assembly 620 does not include an actuator for selectively controlling the flow of fluid through the third fluid inlet 624c (and thus the flow of fluid through the third channel 636c). Instead, the third fluid inlet 624c is configured to remain open/accessible when the system 600 is implanted. Accordingly, fluid can continuously drain through the plate assembly 620 via the third fluid inlet 624c and the third channel 636c when the system 600 is implanted. Without being bound by theory, ensuring a base level of continuous therapy (e.g., flow) may be advantageous in certain circumstances, e.g., such as to ensure that at least a minimum level of therapy is provided. The level of therapy can subsequently be increased or otherwise changed relative to the base level by selectively actuating the first actuator 630a and/or the second actuator 630b, as described in detail above with respect to FIGS. 1A-5.

In some embodiments, such as the illustrated embodiment, the channel that does not have a corresponding actuator and therefore is configured to remain continuously open has the highest fluid resistance of any of the channels. For example, the third channel 636c has a greater length, and therefore greater resistance, than the first channel 636a and the second channel 636b. In other embodiments, the channel that does not have a corresponding actuator can have an equal or even lower fluid resistance than the channels that do have corresponding actuators.

As one skilled in the art will appreciate, the system 600 can include any of the features described with respect to the system 100, such as one or more frictional elements or ledges for reducing the effects of recovery motion of the first actuator 630a and/or the second actuator 630b, and/or one or more sealing elements for improving a seal between the first actuator 630a and the first fluid inlet 624a and/or between the second actuator 630b and the second fluid inlet 624b. The system 100 can also include more or fewer channels through the plate assembly 620, such as two, four, five, six, or more.

Although the present disclosure describes recovery motion in shape memory actuation elements, one skilled in the art will appreciate that this phenomenon can be described in alternative manners. For example, the motion of an actuation element during an actuation event (e.g., a motion toward the actuation element's preferred geometry) can be described as a primary movement of the actuation element. The “recovery motion” of the same actuation element after the actuation event can be described as a secondary movement of the actuation element, as it generally occurs in sequence (e.g., after) the primary movement. Likewise, the movement of the gating element during an actuation event (e.g., movement of the gating element during the primary movement of the actuation element) can be described as a “desired movement” or primary movement of the gating element. Movement of the gating element after the actuation event (e.g., movement of the gating element caused by the secondary movement of the actuation element) can be called an “undesirable movement,” “indirect movement,” and/or a secondary or tertiary movement of the gating element, as it also occurs in sequence (e.g., after) the primary movement of the gating element. Accordingly, one skilled in the art will appreciate that the actuators described herein can demonstrate a series of movements over time during and after an actuation event. The frictional features described herein are expected to permit certain of those movements while mitigating (and/or mitigating the effects of) others. For example, the frictional features generally permit the primary movement of both the actuation element and the gating element, while inhibiting the secondary movement of the gating element that would be ordinarily be caused by the secondary movement of the actuation element.

### EXAMPLES

Several aspects of the present technology are set forth in the following examples:
1. An adjustable shunting system for draining fluid from a first body region to a second body region, the system comprising:
a shunting element configured to extend at least partially between the first body region and the second body region;
an aperture fluidly connecting an exterior of the shunting element to an interior of the shunting element; and
an actuator configured to selectively control fluid flow through the aperture, the actuator having—
   a gating element moveable between a first position in which it does not interfere with fluid flow through the aperture and a second position in which it at least partially blocks fluid flow through the aperture, and
   a shape memory actuation element configured to move the gating element from the first position to and/or toward the second position when actuated; and
a friction element configured to physically engage the gating element to releasably retain the gating element at and/or proximate the second position following actuation of the shape memory actuation element.
2. The system of example 1 wherein the friction element is configured to frictionally engage the gating element.
3. The system of example 1 or 2 wherein the shape memory actuation element is a first shape memory actuation element, the system further comprising a second shape memory actuation element configured to move the gating element from the second position to and/or toward the first position when actuated.
4. The system of example 3 wherein the second shape memory actuation element is configured to overcome the force between the gating element and the friction element to move the gating element from the second position to and/or toward the first position.
5. The system of example 3 or example 4 wherein the friction element is a first friction element, the system further comprising a second friction element configured to engage the gating element to retain the gating element at and/or proximate the first position following actuation of the second shape memory element.
6. The system of any of examples 1-5 wherein the actuator is positioned in a chamber between a first surface having the aperture and a second surface having the friction element.
7. The system of example 6 wherein the friction element includes a ledge on the second surface, and wherein the ledge is aligned with an axis extending through the aperture.
8. The system of example 7 wherein the gating element includes a first surface configured to engage the aperture when in the second position and a second surface configured to engage the ledge when in the second position.
9. The system of example 8 wherein the ledge is further configured to direct the gating element toward the aperture as the gating element moves from the first position toward the second position to improve a contact between the upper surface of the gating element and the aperture.
10. The system of any of examples 7-9 wherein the ledge is wedge-shaped.
11. The system of any of examples 7-10 wherein the ledge is textured.
12. The system of any of examples 1-11 wherein the system includes a plate assembly, the plate assembly having the aperture, the actuator, and the friction element.
13. The system of example 12 wherein the plate assembly includes a plurality of sheets, and wherein the friction element is integral with one of the plurality of sheets.
14. The system of any of examples 1-13 wherein the shape memory actuation element is configured to exhibit a recovery motion following actuation, and wherein the friction element is configured to reduce any undesirable motion of the gating element during the recovery motion of the shape memory actuation element.
15. The system of any of examples 1-14 wherein the system is an intraocular shunting system, the first body region is an anterior chamber of a patient's eye, and the second body region is a desired outflow location.
16. The system of any of examples 1-15 wherein the gating element includes a sealing element configured to abut and/or partially occupy the aperture when the gating element is in the second position to improve a fluidic seal of the aperture.
17. The system of example 16 wherein the sealing element is composed at least partially of a lubricious material and/or includes a lubricious coating.
18. The system of example 16 or example 17 wherein the sealing element is composed at least partially of silicone.
19. The system of any of examples 16-18 wherein the sealing element is composed of a combination of a hydrophobic material and a hydrophilic material.
20. The system of any of examples 16-19 wherein, when in the second position, the gating element has a first surface facing the inlet and a second surface facing away from the inlet, and wherein the sealing element includes a first portion positioned on the first surface configured to abut and/or partially occupy the aperture and a second portion positioned on the second surface and configured to engage the fiction element.
21. The system of example 20 wherein the first portion of the sealing element is composed of a lubricous material and/or includes a lubricious coating.
22. The system of example 21 wherein the second portion of the sealing element is composed of a non-lubricious material.
23. The system of any of examples 1-22 wherein the aperture is a first aperture, the system further comprising:
a second aperture fluidly connecting the exterior of the shunting element to the interior of the shunting element;
a first channel fluidly coupled to the first aperture; and
a second channel fluidly coupled to the second aperture, wherein the second channel is substantially fluidly isolated from the first channel, and wherein the second aperture is free of any corresponding actuator and is configured to remain substantially open when the system is implanted.
24. An adjustable shunting system for draining fluid from a first body region to a second body region, the system comprising:
a shunting element configured to extend at least partially between the first body region and the second body region;
an aperture fluidly connecting an exterior of the shunting element to an interior of the shunting element;
an actuator configured to selectively control fluid flow through the aperture, the actuator having—
   a gating element moveable between a first position in which a first end portion of the gating element does not interfere with fluid flow through the aperture and a second position in which the first end portion of the gating element at least partially interferes with fluid flow through the aperture,
   a first shape memory actuation element configured to move the gating element from the first position to and/or toward the second position when actuated, and
   a second shape memory actuation element configured to move the gating element from the second position to and/or toward the first position when actuated; and
a frictional element configured to physically engage the first end portion of the gating element,
wherein (a) when the gating element is in the first position, the frictional element biases the gating element toward and/or releasably retains the gating element at the first position, and (b) when the gating element is in the second position, the frictional element biases the gating element toward and/or releasably retains the gating element at the second position.
25. The system of example 24 wherein the frictional element is configured to:
permit movement of the gating element from the first position to and/or toward the second position upon actuation of the first shape memory actuation element;
inhibit movement of the gating element from the second position toward the first position after actuation of the first shape memory actuation element;
permit movement of the gating element from the second position to and/or toward the first position upon actuation of the second shape memory actuation element; and
inhibit movement of the gating element from the first position toward the second position after actuation of the second shape memory actuation element.
26. The system of example 24 or 25 wherein the first shape memory actuation element and the second shape memory actuation element are configured to exhibit a recovery motion following actuation, and wherein the frictional element is configured to reduce and/or prevent any undesirable motion of the gating element following actuation of the first actuation element and following actuation of the second actuation element.
27. The system of any of examples 24-26 wherein the first shape memory actuation element is configured to impart a rotational force on the gating element toward the second position when actuated, and wherein the rotational force is greater than a static frictional force between the gating element and the frictional element such that the gating element slides past the frictional element toward the second position.
28. The system of any of examples 24-27 wherein the second shape memory actuation element is configured to impart a rotational force on the gating element toward the first position when actuated, and wherein the rotational force is greater than a static frictional force between the gating element and the frictional element such that the gating element slides past the frictional element toward the first position.
29. The system of any of examples 24-28 wherein the frictional element is integral with the actuator.
30. The system of any of examples 24-28 wherein the frictional element is a tab, nub, projection, or bump.
31. An adjustable shunting system for draining fluid from a first body region to a second body region, the system comprising:
a shunting element configured to extend at least partially between the first body region and the second body region;
an aperture fluidly connecting an exterior of the shunting element to an interior of the shunting element;
an actuator configured to selectively control fluid flow through the aperture, the actuator having—
   a gating element moveable between a first position in which a first end portion of the gating element does not interfere with fluid flow through the aperture and a second position in which the first end portion of the gating element at least partially interferes with fluid flow through the aperture,
   a first shape memory actuation element configured to (1) exhibit a primary movement toward a preferred geometry when actuated that causes the gating element to move from the first position to and/or toward the second position, and (2) exhibit a secondary movement away from its preferred geometry after actuation,
   a second shape memory actuation element configured to (1) exhibit a primary movement toward a preferred geometry when actuated that causes the gating element to move from the second position to and/or toward the first position, and (2) exhibit a secondary movement away from its preferred geometry after actuation,
a frictional element configured to physically engage the first end portion of the gating element,
wherein the frictional element is configured to reduce undesirable motion of the gating element during secondary movement of the first actuation element and during secondary movement of the second actuation element.
32. The system of example 31 wherein the secondary movement of the first actuation element is a recovery motion caused at least in part by the second actuation element, and wherein the secondary movement of the second actuation element is a recovery motion caused at least in part by the first actuation element.
33. An adjustable shunting system for draining fluid from a first body region to a second body region, the system comprising:
a shunting element configured to extend at least partially between the first body region and the second body region;
an aperture fluidly connecting an exterior of the shunting element to an interior of the shunting element;
an actuator configured to selectively control fluid flow through the aperture, the actuator having—
   a gating element,
   a first shape memory actuation element coupled to a first portion of the gating element, and
   a second shape memory actuation element coupled to a second portion of the gating element,
   wherein the first shape memory actuation element is configured to move the gating element from a first low-energy position in which the gating element does not interfere with fluid flow through the aperture through an intermediate high-energy position and to a second low-energy position in which the gating element at least partially interferes with fluid flow through the aperture; and
   wherein the second shape memory actuation element is configured to move the gating element from the second low-energy position through the intermediate high-energy position to the first low-energy position.
34. The system of example 33 wherein the actuator is configured to reduce and/or prevent undesirable motion of the gating element during a recovery motion of the first or second shape memory actuation element by virtue of the intermediate high-energy position.
35. The system of example 34 wherein the actuator is configured such that—
a first force exerted on the gating element by the first shape-memory actuation element during actuation of the first shape-memory actuation element is sufficient to move the gating element through the intermediate high-energy position; and
a second force exerted on the gating element by the first shape-memory actuation element during a recovery motion of the first shape-memory actuation element is not sufficient to move the gating element through the intermediate high-energy position.
36. The system of example 33 or example 34 wherein the gating element has a first internal stress distribution in the first low-energy position, a second internal stress distribution in the second low-energy position, and a third internal stress distribution in the intermediate high-energy position, the gating element being biased toward the first internal stress distribution and/or the second internal stress distribution.
37. The system of example 36 wherein the first internal stress distribution and the second internal stress distribution are approximately equal.
38. A method of adjusting fluid flow through an aperture of a shunt, the method comprising:
actuating a shape memory actuator to move a gating element between (a) a first position that does not interfere with fluid flow through the aperture and (b) a second position that at least partially interferes with fluid flow through the aperture,
wherein moving the gating element between the first position and the second position includes overcoming a first biasing force directing the gating element toward the first position; and
releasably retaining the gating element at the second position during a recovery movement of the shape memory actuator.
39. The method of example 38 wherein releasably retaining the gating element at the second position includes preventing movement of the gating element from the second position toward the first position during the recovery motion of the shape memory actuator.
40. The method of example 38 wherein releasably retaining the gating element at the second position includes engaging the gating element with a frictional element.
41. The method of example 40 wherein the frictional element creates the biasing force.
42. The method of any one of examples 38-41 wherein the gating element has a first stored energy in the first position and the second position, and wherein moving the gating element from the first position to the second position includes transitioning the gating element through an intermediate position having a second stored energy greater than the first stored energy.

### CONCLUSION

The above detailed description of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, any of the features of the intraocular shunts described herein may be combined with any of the features of the other intraocular shunts described herein and vice versa. Moreover, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions associated with intraocular shunts have not been shown or described in detail to avoid unnecessarily obscuring the description of the embodiments of the technology. Where the context permits, singular or plural terms may also include the plural or singular term, respectively.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. An adjustable shunting system for draining fluid from a first body region to a second body region, the system comprising:
a shunting element configured to extend at least partially between the first body region and the second body region;
an aperture fluidly connecting an exterior of the shunting element to an interior of the shunting element; and
an actuator configured to selectively control fluid flow through the aperture, the actuator having—
a gating element moveable between a first position in which it does not interfere with fluid flow through the aperture and a second position in which it at least partially blocks fluid flow through the aperture, and
a shape memory actuation element configured to move the gating element from the first position to and/or toward the second position when actuated; and
a friction element configured to physically engage the gating element to releasably retain the gating element at and/or proximate the second position following actuation of the shape memory actuation element.

2. The system of claim 1 wherein the friction element is configured to frictionally engage the gating element.

3. The system of claim 1 wherein the shape memory actuation element is a first shape memory actuation element, the system further comprising a second shape memory actuation element configured to move the gating element from the second position to and/or toward the first position when actuated.

4. The system of claim 3 wherein the second shape memory actuation element is configured to overcome the force between the gating element and the friction element to move the gating element from the second position to and/or toward the first position.

5. The system of claim 3 wherein the friction element is a first friction element, the system further comprising a second friction element configured to engage the gating element to retain the gating element at and/or proximate the first position following actuation of the second shape memory element.

6. The system of claim 1 wherein the actuator is positioned in a chamber between a first surface having the aperture and a second surface having the friction element.

7. The system of claim 6 wherein the friction element includes a ledge on the second surface, and wherein the ledge is aligned with an axis extending through the aperture.

8. The system of claim 7 wherein the gating element includes a first surface configured to engage the aperture when in the second position and a second surface configured to engage the ledge when in the second position.

9. The system of claim 8 wherein the ledge is further configured to direct the gating element toward the aperture as the gating element moves from the first position toward the second position to improve a contact between the upper surface of the gating element and the aperture.

10. The system of any of claim 7 wherein the ledge is wedge-shaped.

11. The system of any of claim 7 wherein the ledge is textured.

12. The system of claim 1 wherein the system includes a plate assembly, the plate assembly having the aperture, the actuator, and the friction element.

13. The system of claim 12 wherein the plate assembly includes a plurality of sheets, and wherein the friction element is integral with one of the plurality of sheets.

14. The system of claim 1 wherein the shape memory actuation element is configured to exhibit a recovery motion following actuation, and wherein the friction element is configured to reduce any undesirable motion of the gating element during the recovery motion of the shape memory actuation element.

15. The system of claim 1 wherein the system is an intraocular shunting system, the first body region is an anterior chamber of a patient's eye, and the second body region is a desired outflow location.

16. The system of claim 1 wherein the gating element includes a sealing element configured to abut and/or partially occupy the aperture when the gating element is in the second position to improve a fluidic seal of the aperture.

17. The system of claim 16 wherein the sealing element is composed at least partially of a lubricious material and/or includes a lubricious coating.

18. The system of claim 16 wherein the sealing element is composed at least partially of silicone.

19. The system of any of claim 16 wherein the sealing element is composed of a combination of a hydrophobic material and a hydrophilic material.

20. The system of claim 16 wherein, when in the second position, the gating element has a first surface facing the inlet and a second surface facing away from the inlet, and wherein the sealing element includes a first portion positioned on the first surface configured to abut and/or partially occupy the aperture and a second portion positioned on the second surface and configured to engage the fiction element.

21. The system of claim 20 wherein the first portion of the sealing element is composed of a lubricous material and/or includes a lubricious coating.

22. The system of claim 21 wherein the second portion of the sealing element is composed of a non-lubricious material.

23. The system of claim 1 wherein the aperture is a first aperture, the system further comprising:
a first channel fluidly coupled to the first aperture;
a second aperture fluidly connecting the exterior of the shunting element to the interior of the shunting element; and
a second channel fluidly coupled to the second aperture, wherein the second channel is substantially fluidly isolated from the first channel, and wherein the second aperture is free of any corresponding actuator and is configured to remain substantially open when the system is implanted.

24. An adjustable shunting system for draining fluid from a first body region to a second body region, the system comprising:
a shunting element configured to extend at least partially between the first body region and the second body region;
an aperture fluidly connecting an exterior of the shunting element to an interior of the shunting element;
an actuator configured to selectively control fluid flow through the aperture, the actuator having—
a gating element moveable between a first position in which a first end portion of the gating element does not interfere with fluid flow through the aperture and a second position in which the first end portion of the gating element at least partially interferes with fluid flow through the aperture,
a first shape memory actuation element configured to move the gating element from the first position to and/or toward the second position when actuated, and
a second shape memory actuation element configured to move the gating element from the second position to and/or toward the first position when actuated; and
a frictional element configured to physically engage the first end portion of the gating element,
wherein (a) when the gating element is in the first position, the frictional element biases the gating element toward and/or releasably retains the gating element at the first position, and (b) when the gating element is in the second position, the frictional element biases the gating element toward and/or releasably retains the gating element at the second position.

25. The system of claim 24 wherein the frictional element is configured to:
permit movement of the gating element from the first position to and/or toward the second position upon actuation of the first shape memory actuation element;
inhibit movement of the gating element from the second position toward the first position after actuation of the first shape memory actuation element;
permit movement of the gating element from the second position to and/or toward the first position upon actuation of the second shape memory actuation element; and
inhibit movement of the gating element from the first position toward the second position after actuation of the second shape memory actuation element.

26. The system of claim 24 wherein the first shape memory actuation element and the second shape memory actuation element are configured to exhibit a recovery motion following actuation, and wherein the frictional element is configured to reduce and/or prevent any undesirable motion of the gating element following actuation of the first actuation element and following actuation of the second actuation element.

27. The system of claim 24 wherein the first shape memory actuation element is configured to impart a rotational force on the gating element toward the second position when actuated, and wherein the rotational force is greater than a static frictional force between the gating element and the frictional element such that the gating element slides past the frictional element toward the second position.

28. The system of claim 24 wherein the second shape memory actuation element is configured to impart a rotational force on the gating element toward the first position when actuated, and wherein the rotational force is greater than a static frictional force between the gating element and the frictional element such that the gating element slides past the frictional element toward the first position.

29. The system of claim 24 wherein the frictional element is integral with the actuator.

30. The system of claim 24 wherein the frictional element is a tab, nub, projection, or bump.

31. An adjustable shunting system for draining fluid from a first body region to a second body region, the system comprising:
a shunting element configured to extend at least partially between the first body region and the second body region;
an aperture fluidly connecting an exterior of the shunting element to an interior of the shunting element;
an actuator configured to selectively control fluid flow through the aperture, the actuator having—
a gating element moveable between a first position in which a first end portion of the gating element does not interfere with fluid flow through the aperture and a second position in which the first end portion of the gating element at least partially interferes with fluid flow through the aperture,
a first shape memory actuation element configured to (1) exhibit a primary movement toward a preferred geometry when actuated that causes the gating element to move from the first position to and/or toward the second position, and (2) exhibit a secondary movement away from its preferred geometry after actuation,
a second shape memory actuation element configured to (1) exhibit a primary movement toward a preferred geometry when actuated that causes the gating element to move from the second position to and/or toward the first position, and (2) exhibit a secondary movement away from its preferred geometry after actuation,
a frictional element configured to physically engage the first end portion of the gating element,
wherein the frictional element is configured to reduce undesirable motion of the gating element during secondary movement of the first actuation element and during secondary movement of the second actuation element.

32. The system of claim 31 wherein the secondary movement of the first actuation element is a recovery motion caused at least in part by the second actuation element, and wherein the secondary movement of the second actuation element is a recovery motion caused at least in part by the first actuation element.

33. An adjustable shunting system for draining fluid from a first body region to a second body region, the system comprising:
a shunting element configured to extend at least partially between the first body region and the second body region;
an aperture fluidly connecting an exterior of the shunting element to an interior of the shunting element;
an actuator configured to selectively control fluid flow through the aperture, the actuator having—
a gating element,
a first shape memory actuation element coupled to a first portion of the gating element, and
a second shape memory actuation element coupled to a second portion of the gating element,
wherein the first shape memory actuation element is configured to move the gating element from a first low-energy position in which the gating element does not interfere with fluid flow through the aperture through an intermediate high-energy position and to a second low-energy position in which the gating element at least partially interferes with fluid flow through the aperture; and
wherein the second shape memory actuation element is configured to move the gating element from the second low-energy position through the intermediate high-energy position to the first low-energy position.

34. The system of claim 33 wherein the actuator is configured to reduce and/or prevent undesirable motion of the gating element during a recovery motion of the first or second shape memory actuation element by virtue of the intermediate high-energy position.

35. The system of claim 34 wherein the actuator is configured such that—
a first force exerted on the gating element by the first shape-memory actuation element during actuation of the first shape-memory actuation element is sufficient to move the gating element through the intermediate high-energy position; and
a second force exerted on the gating element by the first shape-memory actuation element during a recovery motion of the first shape-memory actuation element is not sufficient to move the gating element through the intermediate high-energy position.

36. The system of claim 33 wherein the gating element has a first internal stress distribution in the first low-energy position, a second internal stress distribution in the second low-energy position, and a third internal stress distribution in the intermediate high-energy position, the gating element being biased toward the first internal stress distribution and/or the second internal stress distribution.

37. The system of claim 36 wherein the first internal stress distribution and the second internal stress distribution are approximately equal.

38. A method of adjusting fluid flow through an aperture of a shunt, the method comprising:
actuating a shape memory actuator to move a gating element between (a) a first position that does not interfere with fluid flow through the aperture and (b) a second position that at least partially interferes with fluid flow through the aperture,
wherein moving the gating element between the first position and the second position includes overcoming a first biasing force directing the gating element toward the first position; and
releasably retaining the gating element at the second position during a recovery movement of the shape memory actuator.

39. The method of claim 38 wherein releasably retaining the gating element at the second position includes preventing movement of the gating element from the second position toward the first position during the recovery motion of the shape memory actuator.

40. The method of claim 38 wherein releasably retaining the gating element at the second position includes engaging the gating element with a frictional element.

41. The method of claim 40 wherein the frictional element creates the biasing force.

42. The method of claim 38 wherein the gating element has a first stored energy in the first position and the second position, and wherein moving the gating element from the first position to the second position includes transitioning the gating element through an intermediate position having a second stored energy greater than the first stored energy.
